(19) United States
(12) Patent Application Publication
(10) Pub. No.: US 2026/0240972 A1
(43) Pub. Date: Aug. 20, 2026

(54) Compositions and Methods for Treating Fentanyl Dependence

(71) Applicant: Babak Behnam, Clarksburg, MD (US)

(72) Inventors: Babak Behnam, Clarksburg, MD (US); Azim Akbarzadehkhiavi, Tehran (IR)

(73) Assignee: Babak Behnam, Clarksburg, MD (US)

(21) Appl. No.: 19/472,769

(22) PCT Filed: Apr. 11, 2024
(86) PCT No.: PCT/US2024/023963

§ 371 Date: Oct. 6, 2025

Related U.S. Application Data
(60) Provisional application No. 63/495,844, filed on Apr. 13, 2023.

(51) Int. Cl. A61K 39/00; A61K 39/385; A61K 47/02

(52) U.S. Cl. CPC A61K 39/0013; A61K 39/385; A61K 47/02

(57) ABSTRACT

This invention provides a composition of matter comprising a plurality of fentanyl-glutaryl-BSA, wherein the average ratio of fentanyl-glutaryl moieties to BSA is at least 7.0. This invention also provides related pharmaceutical compositions, therapeutic methods, prophylactic methods, synthetic methods, and articles of manufacture.

This application claims the benefit of U.S. Provisional Application No. 63/495,844, filed Apr. 13, 2023, the contents of which are incorporated herein by reference.

Throughout this application, various publications are cited. The disclosure of these publications is hereby incorporated by reference into this application to describe more fully the state of the art to which this invention pertains.

### BACKGROUND OF THE INVENTION

Fentanyl is a narcotic drug useful for treating severe pain. It is a synthetic opioid that is up to 50 times stronger than heroin and 100 times stronger than morphine. It is a major contributor to fatal and nonfatal overdoses in the United States.

There are two types of fentanyl: pharmaceutical fentanyl and illicitly manufactured fentanyl (illicit fentanyl). Both of these are synthetic opioids. Pharmaceutical fentanyl is prescribed by doctors to treat severe pain in patients undergoing surgery or afflicted with advanced-stage cancer. Illicit fentanyl is distributed through illegal drug markets for its heroin-like effect.

Fentanyl has a high risk for addiction and dependence. It can cause respiratory distress and death when taken in high doses or when combined with other substances, especially alcohol or other illicit drugs such as heroin or cocaine.

Naloxone can be used to treat an overdose of an opioid such as fentanyl. It should be given to any person who shows signs of an overdose or when an overdose is suspected. Naloxone can be given as a nasal spray or it can be injected into the muscle, under the skin, or into the veins.

### SUMMARY OF THE INVENTION

This invention provides a composition of matter comprising a plurality of fentanyl-glutaryl-bovine serum albumin (BSA), wherein the average ratio of fentanyl-glutarate moieties to BSA is at least 7.0.

This invention also provides a composition of matter comprising a plurality of fentanyl-glutaryl-BSA, wherein (i) the average ratio of fentanyl-glutaryl moieties to BSA is at least 7.0; (ii) the composition is free of EDAC; (iii) the fentanyl-glutaryl-BSA is adsorbed onto a suitable adjuvant; and (iv) the fentanyl-glutaryl-BSA so adsorbed is suspended in a suitable buffer.

This invention further provides a method for treating a subject addicted to fentanyl comprising administering to the subject an effective amount of the present pharmaceutical composition (i.e., a therapeutically effective amount).

This invention also provides a method for inhibiting the onset of fentanyl addiction in a non-addicted subject comprising administering to the subject an effective amount of the present composition (i.e., a prophylactically effect amount).

This invention provides a first synthetic method for making fentanyl-glutaryl-BSA, comprising the following steps:
(a) contacting fentanyl with glutaric acid in the presence of benzene under conditions permitting the formation of fentanyl-glutarate; and
(b) contacting the resulting fentanyl-glutarate with BSA under conditions permitting the formation of fentanyl-glutaryl-BSA, wherein (i) such conditions comprise the presence of tributylamine, dioxane, and isobutyl chloroformate, and (ii) neither step (a) nor step (b) is performed in the presence of EDAC.

This invention provides a second synthetic method for making fentanyl-glutaryl-BSA, comprising contacting fentanyl-glutarate with BSA under conditions permitting the formation of fentanyl-glutaryl-BSA, wherein (i) such conditions comprise the presence of tributylamine, dioxane, and isobutyl chloroformate, and (ii) the contacting is performed in the absence of EDAC.

This invention provides first and second articles of manufacture (also referred to as kits). The first article comprises (a) a composition of matter comprising a plurality of fentanyl-glutaryl-BSA, wherein (i) the average ratio of fentanyl-glutaryl moieties to BSA is at least 7.0; (ii) the composition is free of EDAC; (iii) the fentanyl-glutaryl-BSA is adsorbed onto a suitable adjuvant; and (iv) the fentanyl-glutaryl-BSA so adsorbed is suspended in a suitable buffer, and (b) a label indicating a use for the composition in treating a human subject addicted to fentanyl.

The second article comprises (a) a composition of matter comprising a plurality of fentanyl-glutaryl-BSA, wherein (i) the average ratio of fentanyl-glutaryl moieties to BSA is at least 7.0; (ii) the composition is free of EDAC; (iii) the fentanyl-glutaryl-BSA is adsorbed onto a suitable adjuvant; and (iv) the fentanyl-glutaryl-BSA so adsorbed is suspended in a suitable buffer, and (b) a label indicating a use for the composition in inhibiting the onset of fentanyl addiction in a non-addicted human subject.

### BRIEF DESCRIPTION OF THE FIGURES

FIG. 1

This figure shows (i) the synthesis of fentanyl-glutarate, and (ii) synthesis of fentanyl-glutaryl-BSA (also referred to as fentanyl-BSA and fentanyl-glutarate-NH-BSA). The role of the dioxane, triethylamine and isobutyl chloroformate in this second reaction is to bind molecules of fentanyl-glutarate to BSA.

FIG. 2

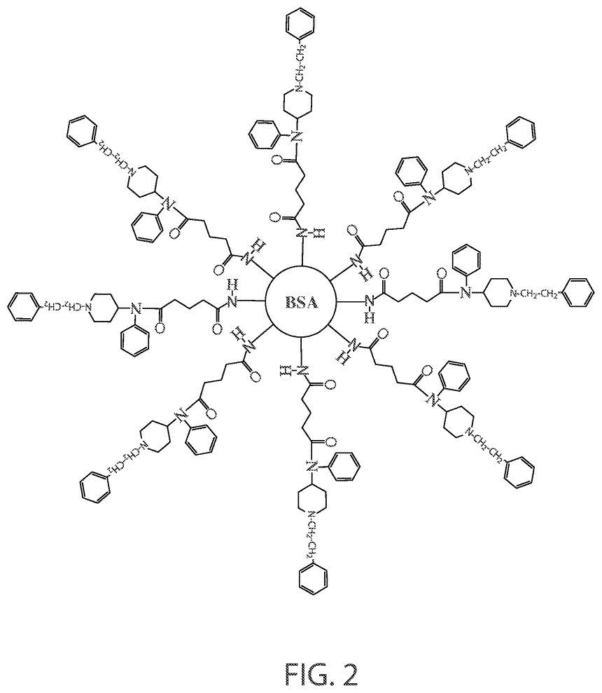

This figure shows a schematic of eight molecules of fentanyl-glutarate conjugated with bovine serum albumin to form a fentanyl vaccine.

### DETAILED DESCRIPTION OF THE INVENTION

### Definitions

In this application, certain terms are used which shall have the meanings set forth as follows.

As used herein, “administer”, with respect to an agent, means to deliver the agent to a subject's body via any known method. Specific modes of administration include, without limitation, intravenous, intramuscular, oral, sublingual, transdermal, subcutaneous, intraperitoneal, and intrathecal administration. Preferred in this invention is intramuscular administration (e.g., injection into the deltoid muscle).

In addition, in this invention, the various agents (e.g., fentanyl-glutaryl-BSA) used can be formulated using one or more routinely used pharmaceutically acceptable carriers appropriate for protein agents. Such carriers are well known to those skilled in the art. For example, injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone. Also included in injectable drug delivery systems are adjuvants (e.g., analgesic adjuvants; inorganic compounds (e.g., alum, aluminum hydroxide, aluminum phosphate, and calcium phosphate); mineral oil (e.g., paraffin oil); bacterial products (killed bacteria Bordetella pertussis, Mycobacterium bovis, toxoids; nonbacterial organics (e.g., squalene); plant saponins from Quillaja, soybean, polygala senega; cytokines (e.g., IL-1, IL-2, and IL-12); Freund's complete adjuvant; and Freund's incomplete adjuvant) and buffers (e.g., phosphate buffered saline) or other diluents such as sterile water.

As used herein, an “effective amount” of the present pharmaceutical composition used in the present prophylactic and therapeutic methods is an amount sufficient to deliver to the subject a prophylactic or therapeutic amount of the fentanyl-glutaryl-BSA (“active agent”) therein, as appropriate. In one embodiment, an effective amount of the present pharmaceutical composition contains an amount of active agent sufficient to deliver from 1 μg to 1,000 μg of active agent to the subject per dose (e.g., 1 μg, 2.5 μg, 5 μg, 7.5 μg, 10 μg, 12.5 μg, 15 μg, 20 μg, 50 μg, 100 μg, 200 μg, 300 μg, 400 μg, 500 μg, 600 μg, 700 μg, 800 μg, 900 μg, or 1,000 μg; or 1-10 μg, 10-20 μg, 20-50 μg, 50-100 μg, 100-500 μg, or 500-1,000 μg). Moreover, this dose can be administered once, or preferably a plurality of times (ideally three) over the course of prophylaxis or therapy.

As used herein, a composition is “free of” EDAC if, for example, it contains less than 1.0% of EDAC, less than 0.9% of EDAC, less than 0.8% of EDAC, less than 0.7% of EDAC, less than 0.6% of EDAC, less than 0.5% of EDAC, less than 0.4% of EDAC, less than 0.3% of EDAC, less than 0.2% of EDAC, less than 0.1% of EDAC, less than 0.05% of EDAC, less than 0.04% of EDAC, less than 0.03% of EDAC, less than 0.02% of EDAC, less than 0.01% of EDAC, or less than 0.001% of EDAC. If a reaction step is not “performed in the presence of EDAC”, the conditions for performing that step are “free of” EDAC. Similarly, a composition is “free of” any amidine if, for example, it contains less than 1.0% of any amidine, less than 0.9% of any amidine, less than 0.8% of any amidine, less than 0.7% of any amidine, less than 0.6% of any amidine, less than 0.5% of any amidine, less than 0.4% of any amidine, less than 0.3% of any amidine, less than 0.2% of any amidine, less than 0.1% of any amidine, less than 0.05% of any amidine, less than 0.04% of any amidine, less than 0.03% of any amidine, less than 0.02% of any amidine, less than 0.01% of any amidine, or less than 0.001% of any amidine. If a reaction step is not “performed in the presence an amidine”, the conditions for performing that step are “free of” any amidine.

As used herein, a “non-addicted” subject, with respect to fentanyl, includes, without limitation, (i) a subject who has never taken fentanyl, and (ii) a subject who has taken fentanyl previously (e.g., medically or recreationally) yet is not addicted (as that term is defined herein).

As used herein, a “human subject” can be of any age, gender, or state of co-morbidity. In one embodiment, the subject is male, and in another, the subject is female.

As used herein, a fentanyl-“addicted” subject is one who craves fentanyl and, if presented with an opportunity to take fentanyl, is incapable of resisting that opportunity absent medical or therapeutic intervention.

As used herein, “inhibiting the onset” of fentanyl addiction in a non-addicted subject includes, without limitation, reducing the likelihood of addiction by at least 10%, at least 20%, at least 30%, at least 40%, at least 50%, at least 60%, at least 70%, at least 80%, at least 90%, or at least 95%. Preferably, inhibiting the onset of fentanyl addiction in a non-addicted subject means preventing such onset.

As used herein, the term “subject” includes, without limitation, a mammal such as a human, a non-human primate, a dog, a cat, a horse, a sheep, a goat, a cow, a rabbit, a pig, a hamster, a rat, and a mouse.

As used herein, “treating” a subject addicted to fentanyl and symptomatic of that addiction includes, without limitation, (i) slowing, stopping, or reversing the progression of one or more symptoms of addiction, (ii) reducing or eliminating the addiction, (iii) reducing or eliminating the likelihood of the symptoms' recurrence, and/or (iv) reducing the likelihood of, or preventing, the addiction's recurrence. In the preferred embodiment, treating a subject addicted to fentanyl and symptomatic of that addiction includes (i) reversing the progression of one or more of the symptoms, (ii) eliminating the addiction, (iii) preventing the symptoms' recurrence, and/or (iv) preventing the addiction's recurrence. The progress of treating a subject addicted to fentanyl and symptomatic of that addiction can be measured according to a number of clinical endpoints. These endpoints include, without limitation, amelioration of one or more of the following: drowsiness; sudden fatigue; dizziness; nausea and vomiting; swollen hands or feet; constricted pupils; insomnia; hyperactivity; incoordination; sweating; muscle aches; inattention; impaired judgment; suicidal ideation; depression; inability to walk; clammy skin; slurred speech or inability to speak; disorientation; limp body; and slow breathing rate.

### EMBODIMENTS OF THE INVENTION

This application provides compositions having use as vaccines for patients dependent on fentanyl, or at risk of becoming dependent thereon. These compositions are unexpectedly superior because, among other reasons, they comprise fentanyl-glutaryl-BSA having advantageously high ratios of hapten to BSA, and are free of EDAC and other harmful amidines.

Specifically, this invention provides a composition of matter comprising a plurality of fentanyl-glutaryl-BSA, wherein the average ratio of fentanyl-glutaryl moieties to BSA is at least 7.0.

In one embodiment, the present composition is free of the amidine 1-ethyl-3-(3-dimethylaminopropyl) carbodiimide (EDAC). Preferably, the present composition is also free of any amidine.

In one embodiment of the present composition, the average ratio of fentanyl-glutaryl moieties to BSA is selected from the group consisting of at least 7.5, at least 8.0, and preferably at least 8.2. By way of example, in the present composition, the average ratio of fentanyl-glutaryl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the present composition, the average ratio of fentanyl-glutaryl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the present composition, the average ratio of fentanyl-glutaryl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

In a preferred embodiment, the present composition comprises a plurality of fentanyl-glutaryl-BSA, wherein (i) the average ratio of fentanyl-glutaryl moieties to BSA is at least 8.2; and (ii) the composition is free of EDAC (and is preferably free of any amidine).

This invention also provides a composition of matter (“pharmaceutical composition”) comprising a plurality of fentanyl-glutaryl-BSA, wherein (i) the average ratio of fentanyl-glutaryl moieties to BSA is at least 7.0; (ii) the composition is free of EDAC; (iii) the fentanyl-glutaryl-BSA is adsorbed onto a suitable adjuvant; and (iv) the fentanyl-glutaryl-BSA so adsorbed is suspended in a suitable buffer. Preferably, the composition is also free of any amidine.

In one embodiment of the present pharmaceutical composition, the average ratio of fentanyl-glutaryl moieties to BSA is selected from the group consisting of at least 7.5, at least 8.0, and preferably at least 8.2. By way of example, in the present pharmaceutical composition, the average ratio of fentanyl-glutaryl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the present pharmaceutical composition, the average ratio of fentanyl-glutaryl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the present pharmaceutical composition, the average ratio of fentanyl-glutaryl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

Adjuvants suitable for use in the present pharmaceutical composition are known. In a preferred embodiment, the suitable adjuvant is aluminum hydroxide gel. Similarly, buffers suitable for use in the present pharmaceutical composition are known. In a preferred embodiment, the suitable buffer is phosphate buffered saline.

In a preferred embodiment, the present pharmaceutical composition comprises a plurality of fentanyl-glutaryl-BSA, wherein (i) the average ratio of fentanyl-glutaryl moieties to BSA is at least 8.2; (ii) the composition is free of EDAC (and preferably is free of any amidine); (iii) the fentanyl-glutaryl-BSA is adsorbed onto aluminum hydroxide gel; and (iv) the fentanyl-glutaryl-BSA so adsorbed is suspended in phosphate buffered saline.

This invention further provides a method for treating a subject addicted to fentanyl comprising administering to the subject an effective amount of the present pharmaceutical composition (i.e., a therapeutically effective amount). Preferably, the subject is human.

In the present therapeutic method, the present pharmaceutical composition can be administered according to any appropriate dosing regimen. In one embodiment, the present pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more “boosters” at suitable intervals (e.g., every two or three years).

In a preferred embodiment of the present therapeutic method, the method comprises injecting the subject with an effective amount of the present pharmaceutical composition, wherein the composition is administered via three doses at 30-day intervals. Preferably, the subject is human.

This invention also provides a method for inhibiting the onset of fentanyl addiction in a non-addicted subject comprising administering to the subject an effective amount of the present composition (i.e., a prophylactically effect amount). Preferably, the subject is human.

In the present prophylactic method, the present pharmaceutical composition can be administered according to any appropriate dosing regimen. In one embodiment, the present pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30, and day 60). In yet another embodiment, this administration is followed with one or more “boosters” at suitable intervals (e.g., every two or three years).

In a preferred embodiment of the present prophylactic method, the method comprises injecting the human subject with an effective amount of the present prophylactic composition, wherein the composition is administered via three doses at 30-day intervals.

The present composition (as opposed to the present pharmaceutical composition) is also envisioned, mutatis mutandis, for use in the present therapeutic and prophylactic methods.

This invention provides a first synthetic method for making fentanyl-glutaryl-BSA, comprising the following steps:
(a) contacting fentanyl with glutaric acid in the presence of benzene under conditions permitting the formation of fentanyl-glutarate; and
(b) contacting the resulting fentanyl-glutarate with BSA under conditions permitting the formation of fentanyl-glutaryl-BSA, wherein (i) such conditions comprise the presence of tributylamine, dioxane, and isobutyl chloroformate, and (ii) neither step (a) nor step (b) is performed in the presence of EDAC.

In a preferred embodiment of the first synthetic method, neither step (a) nor step (b) is performed in the presence of an amidine.

This invention provides a second synthetic method for making fentanyl-glutaryl-BSA, comprising contacting fentanyl-glutarate with BSA under conditions permitting the formation of fentanyl-glutaryl-BSA, wherein (i) such conditions comprise the presence of tributylamine, dioxane, and isobutyl chloroformate, and (ii) the contacting is performed in the absence of EDAC.

In a preferred embodiment of the second synthetic method, the contacting is performed in the absence of any amidine.

This invention provides first and second articles of manufacture (also referred to as kits). The first article comprises (a) a composition of matter comprising a plurality of fentanyl-glutaryl-BSA, wherein (i) the average ratio of fentanyl-glutaryl moieties to BSA is at least 7.0; (ii) the composition is free of EDAC; (iii) the fentanyl-glutaryl-BSA is adsorbed onto a suitable adjuvant; and (iv) the fentanyl-glutaryl-BSA so adsorbed is suspended in a suitable buffer, and (b) a label indicating a use for the composition in treating a human subject addicted to fentanyl.

In a preferred embodiment, the first article comprises (a) a composition of matter comprising a plurality of fentanyl-glutaryl-BSA, wherein (i) the average ratio of fentanyl-glutaryl moieties to BSA is at least 8.2; (ii) the composition is free of EDAC; (iii) the fentanyl-glutaryl-BSA is adsorbed onto aluminum hydroxide gel; and (iv) the fentanyl-glutaryl-BSA so adsorbed is suspended in phosphate buffered saline, and (b) a label indicating a use for the composition in treating a human subject addicted to fentanyl.

The second article comprises (a) a composition of matter comprising a plurality of fentanyl-glutaryl-BSA, wherein (i) the average ratio of fentanyl-glutaryl moieties to BSA is at least 7.0; (ii) the composition is free of EDAC; (iii) the fentanyl-glutaryl-BSA is adsorbed onto a suitable adjuvant; and (iv) the fentanyl-glutaryl-BSA so adsorbed is suspended in a suitable buffer, and (b) a label indicating a use for the composition in inhibiting the onset of fentanyl addiction in a non-addicted human subject.

In a preferred embodiment, the second article comprises (a) a composition of matter comprising a plurality of fentanyl-glutaryl-BSA, wherein (i) the average ratio of fentanyl-glutaryl moieties to BSA is at least 8.2; (ii) the composition is free of EDAC; (iii) the fentanyl-glutaryl-BSA is adsorbed onto aluminum hydroxide gel; and (iv) the fentanyl-glutaryl-BSA so adsorbed is suspended in phosphate buffered saline, and (b) a label indicating a use for the composition in inhibiting the onset of fentanyl addiction in a non-addicted human subject.

Preferably, in the first and second articles, the composition is free of any amidine.

This invention will be better understood by reference to the examples which follow, but those skilled in the art will readily appreciate that the specific examples detailed are only illustrative of the invention as described more fully in the claims which follow thereafter.

### Examples

### Example 1—Production of Fentanyl Glutathione (F-G) Hapten-Carrier Vaccine

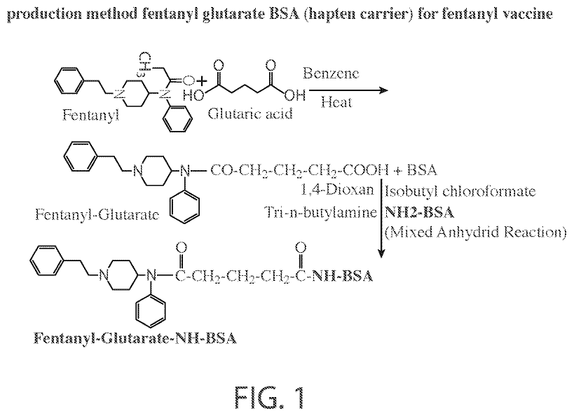

In a 100-ml glass beaker, 0.85 grams (2.15 millimoles) of fentanyl glutarate and 0.5 milliliters (0.4 grams, 2.15 millimoles) of tri-n-butylamine are mixed in 21.5 milliliters (22.15 grams, 251.3 millimoles) of dioxane using the mixed anhydride method. After reducing the temperature of the mixture to 10 degrees Celsius, 0.27 milliliters (0.284 grams, 2.07 millimoles) of iso-butyl chloroformate is added to it. The reaction is then continued for 20 minutes at a temperature of 4 degrees Celsius (FIG. 1). Next, the mixture all is added at once to a 500-milliliter beaker of solution cooled to 5 degrees Celsius, containing a solution of 3.58 grams (0.051 millimoles) of BSA dissolved in 186 milliliters of 1:1 water-dioxane (spectral grade) and 3.58 milliliters of normal NaOH, while stirring the mixture well in a half-liter glass beaker. The addition of the solution causes it to become slightly cloudy and gas bubbles appear. Then, the pH of the solution is adjusted to 7.5 with normal NaOH. After an hour, the pH of the solution is adjusted to 8.5 with normal NaOH. Stirring the mixture is continued for 4 hours at a temperature of 5 degrees Celsius. Finally, the resulting product is poured into a dialysis bag with a cutoff of 30,000 Dalton and dialyzed overnight under a low flow of tap water.

The product is then transferred to a one-liter bottle and the pH is adjusted to 4.5 with normal HCl. The hapten-carrier, fentanyl-glutarate-BSA, starts to precipitate. The precipitate is kept in the one-liter bottle in the refrigerator at 4 degrees Celsius overnight and separated the next day using centrifugation. The hapten precipitate is dispersed in 100 milliliters of distilled water as a suspension and the pH is adjusted to 5.5 with a 0.1 normal NaHCO₃ solution. The product is dialyzed under running water for 4 hours. The yield is 2.34 grams of hapten-carrier, which is equivalent to 45,800 doses of vaccine. It is dissolved in 15 liters of pyrogen-free distilled water and filtration and sterilization continue. 15,800 doses are taken for quality control, waste, storage, and expiration. 30,000 doses are then prepared for the clinical trial phases.

### Example 2—Fentanyl Vaccine Formulation (Fentanyl-BSA)

| | Formulation values | |
|---|---|---|
| | of a 1 ml single | |
| Components Formulation | dose (F.V.) vial | |
| Antigenic protein (fentanyl-BSA) | 50 | ug |
| Aluminum hydroxide (Al³⁺) | 0.5 | mg |
| Sodium chloride | 8.0 | mg |
| Dibasic sodium phosphate anhydrous | 1.12 | mg |
| Monobasic sodium phosphate monohydrate | 1.10 | mg |
| Water for injection Qs | 1.0 | ml |

### REFERENCES

1. A. Akbarzadeh, et al., Asian J. of Biochemistry, 2(1):58-65 (2007).
2. A. Akbarzadeh, et al., J. of Pharm. And Toxicol., 4(1):3035 (2009).
3. A. Akbarzadeh, et al., Biotechnology and Applied Biochem., (Dec. 23, 2010).
4. F. Li, et al., Org. Biomol. Chem. 2014 Oct. 7; 12(37): 7211-7232.
5. M. Pravetoni, et al., Biochem. Pharmacol. 2012 Feb. 15; 83(4):543-50.
6. M. Pravetoni, et al., J. Pharmacol. Exp. Ther. 2012 April; 341(1):225-32.
7. Y. Hu, et al., Hum. Vaccin. Immunother. 2014; 10(1):64-72.
8. S. D. Comer and C. M. Cahill, “Fentanyl: Receptor pharmacology, abuse potential, and implications for treatment”, Neuroscience & Biobehavioral Reviews, Vol. 106, November 2019, pp. 49-57.

1.-34. (canceled)

35. A composition of matter comprising a plurality of fentanyl-glutaryl-bovine serum albumin (BSA), wherein the average ratio of fentanyl-glutaryl moieties to BSA is selected from the group consisting of at least 7.0, at least 7.5, at least 8.0, and at least 8.2.

36. The composition of claim 35, wherein the composition is free of 1-ethyl-3-(3-dimethylaminopropyl) carbodiimide (EDAC), and optionally free of any amidine.

37. The composition of claim 35, wherein (i) the composition is free of EDAC;
(ii) the fentanyl-glutaryl-BSA is adsorbed onto a suitable adjuvant; (iii) the fentanyl-glutaryl-BSA so adsorbed is suspended in a suitable buffer; and (iv) the composition is optionally free of any amidine.

38. The composition of claim 37, wherein the suitable adjuvant is aluminum hydroxide gel.

39. The composition of claim 37, wherein the suitable buffer is phosphate buffered saline.

40. A method for treating a subject addicted to fentanyl comprising administering to the subject an effective amount of the composition of claim 35.

41. The method of claim 40, wherein the subject is human.

42. A method for treating a human subject addicted to fentanyl comprising injecting the subject with an effective amount of the composition of claim 37, wherein the composition is administered via three doses at 30-day intervals.

43. A method for inhibiting the onset of fentanyl addiction in a non-addicted subject comprising administering to the subject an effective amount of the composition of claim 37.

44. The method of claim 43, wherein the subject is human.

45. A method for inhibiting the onset of fentanyl addiction in a non-addicted human subject comprising injecting the subject with an effective amount of the composition of claim 37, wherein the composition is administered via three doses at 30-day intervals.

46. A method for making fentanyl-glutaryl-BSA, comprising the following steps (a) and (b):
(a) contacting fentanyl with glutaric acid in the presence of benzene under conditions permitting the formation of fentanyl-glutarate; and
(b) contacting the resulting fentanyl-glutarate with BSA under conditions permitting the formation of fentanyl-glutaryl-BSA, wherein (i) such conditions comprise the presence of tributylamine, dioxane, and isobutyl chloroformate, and (ii) neither step (a) nor step (b) is performed in the presence of EDAC, or
alternatively comprising the following step (c):
(c) contacting fentanyl-glutarate with BSA under conditions permitting the formation of fentanyl-glutaryl-BSA, wherein (i) such conditions comprise the presence of tributylamine, dioxane, and isobutyl chloroformate, and (ii) the contacting is performed in the absence of EDAC.

47. An article of manufacture comprising (ai) the composition of claim 35, and (b) a label indicating a use for the composition in treating a human subject addicted to fentanyl.

48. An article of manufacture comprising (a) the composition of claim 37, and (b) a label indicating a use for the composition in treating a human subject addicted to fentanyl.

49. An article of manufacture comprising (a) the composition of claim 35, and (b) a label indicating a use for the composition in inhibiting the onset of fentanyl addiction in a non-addicted human subject.

50. An article of manufacture comprising (a) the composition of claim 37, and (b) a label indicating a use for the composition in inhibiting the onset of fentanyl addiction in a non-addicted human subject.
